(19) United States
(12) Patent Application Publication
(10) Pub. No.: US 2020/0114016 A1
(43) Pub. Date: Apr. 16, 2020

(54) Anti-Warburg Nanoparticles - A matrix metalloprotease activatable conjugate to inhibit glioblastoma proliferation

(71) Applicant: The Board of Trustees of the Leland Stanford Junior University, Stanford, CA (US)

(72) Inventors: Heike E. Daldrup-Link, San Francisco, CA (US); Suchismita Mohanty, Burlingame, CA (US)

(21) Appl. No.: 16/615,514

(22) PCT Filed: May 23, 2018
(86) PCT No.: PCT/US2018/034217

§ 371 Date: Nov. 21, 2019

Related U.S. Application Data
(60) Provisional application No. 62/509,855, filed on May 23, 2017.

(51) Int. Cl. A61K 47/65; A61K 49/18; A61K 47/69; A61K 31/19

(52) U.S. Cl. CPC A61K 47/65; A61K 31/19; A61K 47/6923; A61K 49/1866

(57) ABSTRACT

A matrix metalloprotease (MMP) activatable conjugate is provided to inhibit glioblastoma (GBM) proliferation through glioma tumor cell and glioma stem cell differentiation. The conjugate is structurally characterized by having dichloroacetate (DCA), as a therapeutic drug, linked, via a MMP cleavable peptide, to a nanoparticle suitable for magnetic resonance imaging (MRI) such as a superparamagnetic iron oxide (SIPO) or an ultra superparamagnetic iron oxide (uSIPO).

### FIELD OF THE INVENTION

This invention relates to conjugates and methods of changing glioblastoma cells into a different cell type to prevent replication.

### BACKGROUND OF THE INVENTION

Glioblastoma (GBM) is the most frequently diagnosed primary malignant brain tumor in children and adults with median survival of less than one year. GBM invariably progresses because it contains glioma stem cells (GSCs), which are resistant to classical chemotherapy and continue to renew and expand the tumor. Pre-clinical and clinical experience over the past 2 decades has shown that attempting to kill GSC with cytotoxic drugs does not work. Traditional combination therapy with surgery, radiation and chemotherapy has only slightly increased median survival times from 12-15 months. We urgently need a different approach.

### SUMMARY OF THE INVENTION

The present invention provides a matrix metalloprotease (MMP) activatable conjugate to inhibit glioblastoma (GBM) proliferation through glioma tumor cell and glioma stem cell differentiation. The conjugate is structurally characterized by having dichloroacetate (DCA), as a therapeutic drug, which induces glioblastoma (GMB) differentiation and stops tumor growth by inhibiting pyruvate dehydrogenase, reactivating ATP production in mitochondria, lowering the glycolytic flux of cancer cells, reducing the incorporation of glucose, and reducing the activity of biosynthetic pathways such as the pentose phosphate pathway.

The conjugate is further structurally characterized by a nanoparticle which has an ion and is a magnetic resonance imaging (MRI) contrast agent. Examples of the ion are: gadolinium, iron, platinum, manganese, copper, gold or barium. Another example of the ion is an iron oxide. Specific examples of the nanoparticle are a superparamagnetic iron oxide (SIPO) or ultra-superparamagnetic iron oxide (uSIPO).

An MMP cleavable peptide links the DCA and the nanoparticle formulating the matrix metalloprotease (MMP) activatable conjugate to inhibit glioblastoma (GBM) proliferation. Examples of MMP cleavable peptides are, for example, mentioned in a paper by Ansari et al. 2013 entitled Development of Novel Tumor-Targeted Theranostic Nanoparticles Activated by Membrane-Type Matrix Metalloproteinases for Combined Cancer Magnetic Resonance Imaging and Therapy and published in Small. 2014 Feb. 12; 10(3): 566-417.

The matrix metalloprotease (MMP) activatable conjugate according to this invention does not comprise folate and/or does not comprise a vascular disrupting agent (VDA).

The matrix metalloprotease (MMP) activatable conjugate according to this invention has the following advantages:
(1) DCA induces GBM cell differentiation as opposed to other cytotoxic drugs that cause systemic toxicity.
(2) The conjugate is specifically activated in tumor tissue with tumor enzyme MMPs thereby preventing systemic toxicity.
(3) The conjugate can be activated inherently in tumor tissue without the need for any external stimulus e.g. such as near-infrared light, radiofrequency ablation, or thermal induction e.g. such as near-infrared light, radiofrequency ablation, or thermal induction
(4) DCA-conjugated nanoparticles are smaller than 100 nm their undesirable accumulation in liver and spleen is attenuated. Previously tested, larger iron oxide nanoparticles demonstrated marked uptake by the reticuloendothelial system with inefficient tumor delivery
(5) Our nanoparticles are based on FDA-approved nanoparticles and detectable with clinically applicable MR imaging approaches, which will facilitate clinical translation.

### BRIEF DESCRIPTION OF THE DRAWINGS

FIG. 1 shows according to an exemplary embodiment of the invention the principle of anti-Warburg theranostic nanoparticles.

FIG. 2 shows according to an exemplary embodiment of the invention the chemical steps performed for synthesis of matrix metalloprotease (MMP) activatable anti-Warburg theranostic nanoparticles.

FIG. 3A shows according to an exemplary embodiment of the invention anti-tumor efforts of dichloroacetate (DCA)—Phase contrast images of pcGMB39 cells treated with Phosphate Buffer Saline (PBS) or DCA for 72 hours.

FIG. 3B shows according to an exemplary embodiment of the invention anti-tumor efforts of dichloroacetate (DCA)—Dose-dependent effect of DCA on glioblastoma (GBM) cell viability. An MTT (3-(4,5-dimethylthiazol-2-yl)-2,5-diphenyltetrazolium bromide) assay was used to determine cell viability.

FIG. 3C shows according to an exemplary embodiment of the invention anti-tumor efforts of dichloroacetate (DCA)—q-PCR (Quantitative-PCR) expression of MMP-14 in normal brain cortical cells (HCN2) and in glioblastoma (GBM) cell lines.

FIG. 3D shows according to an exemplary embodiment of the invention anti-tumor efforts of dichloroacetate (DCA)—immunofluorescence images for MMP-14 expression in glioblastoma (GBM) cell lines versus normal brain. Scale bar 75 microns. Results represented as mean±SD from three independent experiments * p<0.05, ** p<0.005, one-way ANOVA.

FIG. 4A shows according to an exemplary embodiment of the invention delivery of nanoparticle conjugated therapeutic drug in GBM. Representative T₂ weighted MR images of mice brain. T₂ FSE sequences were used to capture coronal T₂ weighted images. Nanoparticle and theranostic nanoparticle delivery is demonstrated by T₂ darkening or negative enhancement (arrowheads) in CLIO and CLIO-ICT treated animals respectively. CLIO stands for cross-linked iron oxide nanoparticles. ICT is a peptide-conjugate of azademethylcolchicine (ICT2552), which is selectively cleavable by MMP-14 to release the active VDA azademethylcolchicine vasculature disruptive agent. CLIO-ICT is CLIO nanoparticles linked to ICT. PBS stand for phosphate buffer saline.

FIG. 4B shows according to an exemplary embodiment of the invention anti-tumor effects of nanoparticle conjugated therapeutic drug in GBM. Bioluminescent in vivo images of tumors in mice treated with CLIO-ICT (0.5 mmol Fe/kg and 20 mg/kg of ICT), ICT (20 mg/kg of ICT), CLIO (0.5 mmol Fe/kg) or vehicle.

FIG. 4C shows according to an exemplary embodiment of the invention anti-tumor effects of nanoparticle conjugated therapeutic drug in GBM. Kaplan-Meyer survival curves of control and treated mice demonstrate a significant survival benefit of CLIO-ICT as compared to vehicle, log-rank Mantel-Cox test.

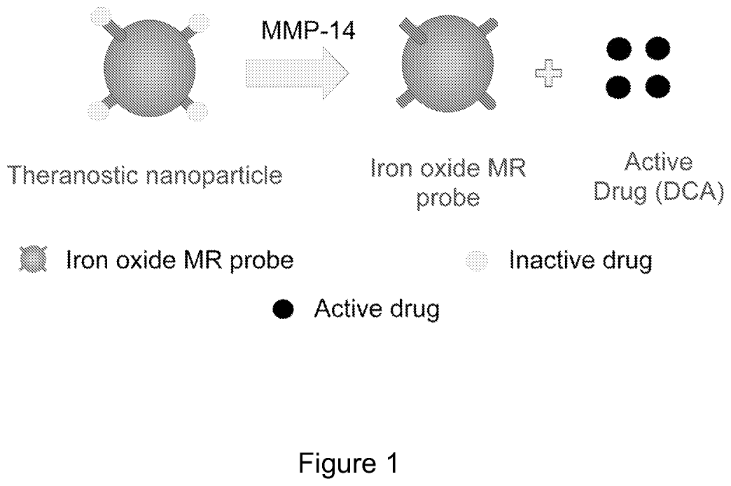

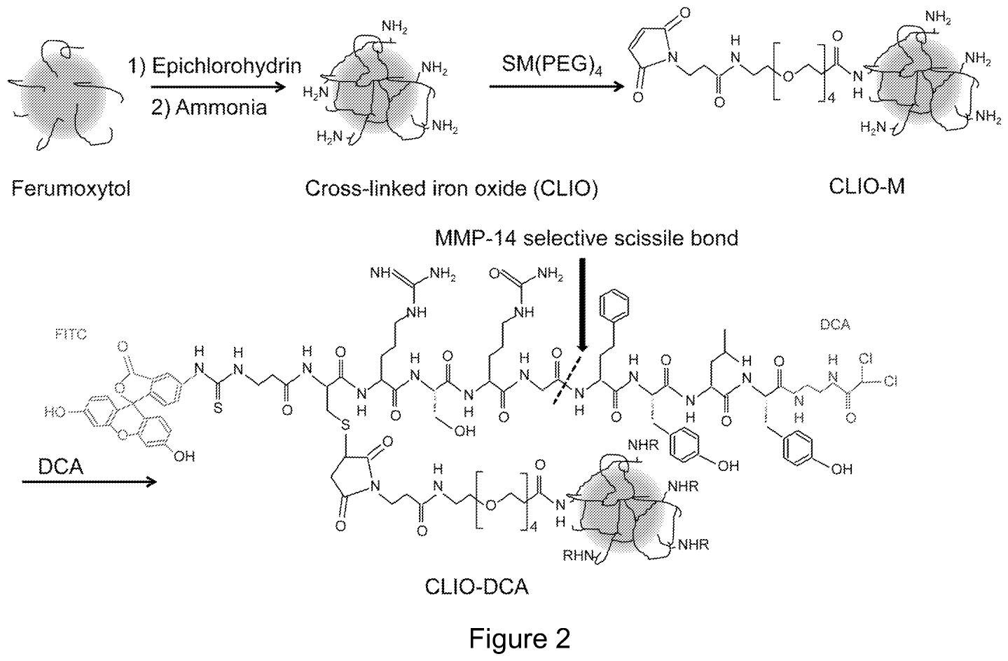

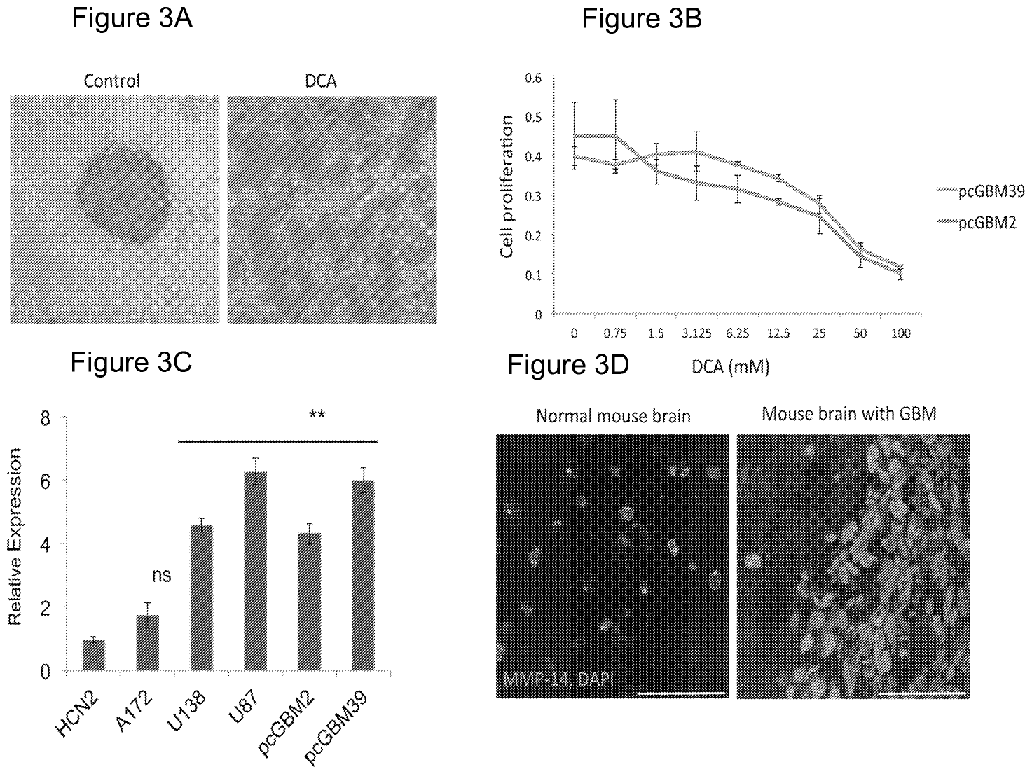

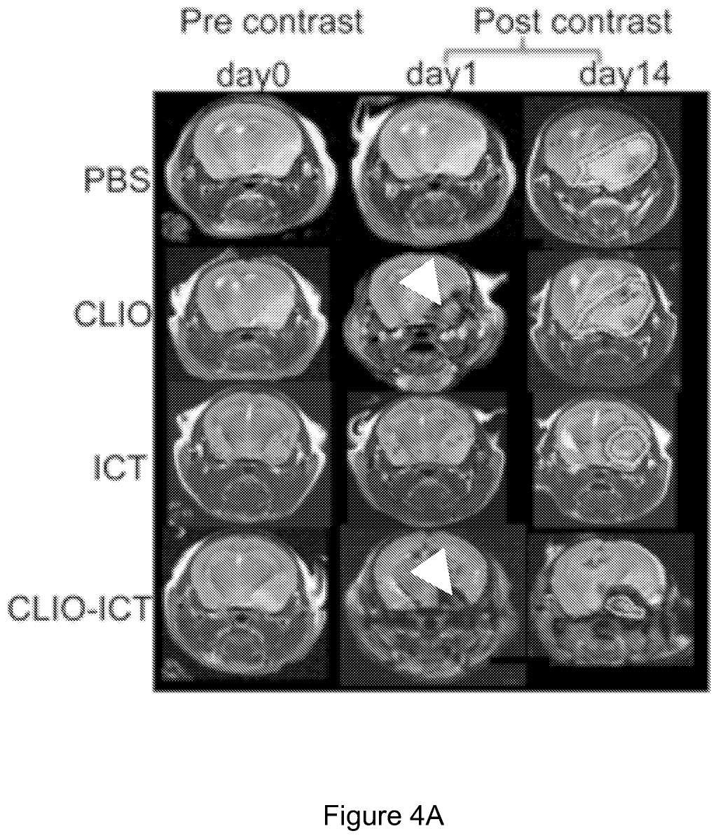

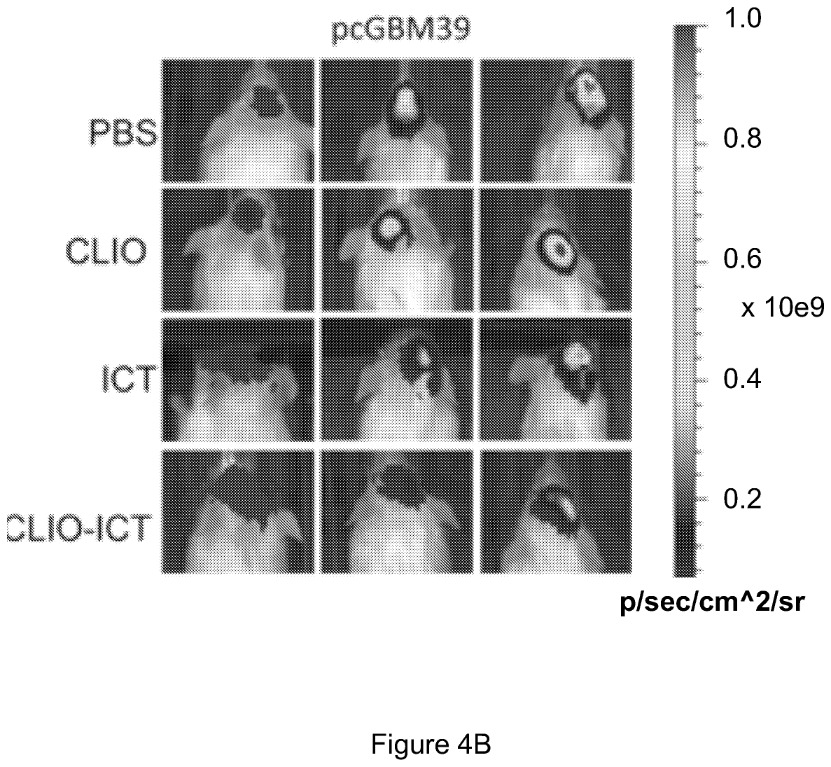

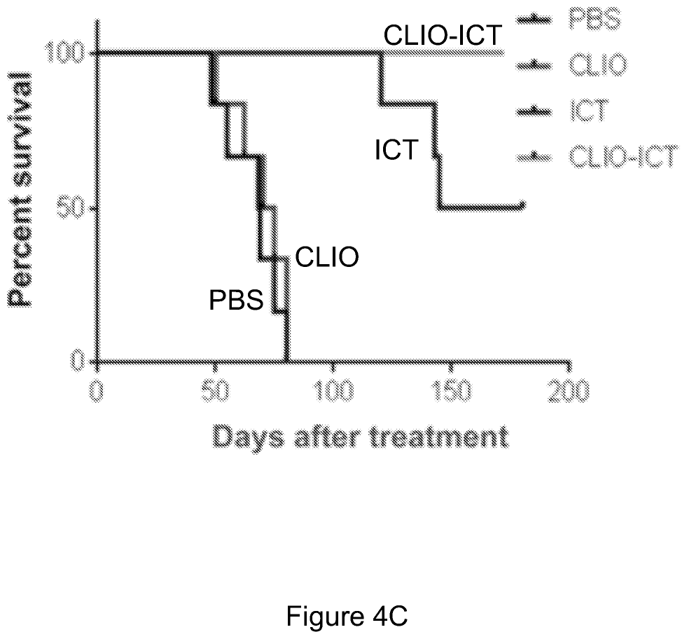

### DETAILED DESCRIPTION

With this invention we provide a new and different approach. Instead of trying to intoxicate continuously renewing GSC, we transform GCS into a different cell type, which cannot replicate. This can be achieved by forced differentiation of GSCs to neuronal cell lineages.

Differentiating rather than killing cancer cells has demonstrated significant clinical benefits in the treatment of hematologic malignancies. However, differentiation of GSC has not been achieved to date. During gliomagenesis, a nutrient-restricted microenvironment leads a “metabolic switch” (Warburg effect) in GSCs with enhanced glycolytic flux, faster production of ATP and markedly accelerated cell proliferation. The Warburg effect is turned “off” in normal brain cells, such as neurons and glia cells.

It has been shown that molecular targets that shift tumor metabolism from glycolysis to oxidative phosphorylation deplete ATP pools, direct the differentiation of GBM cells to astrocytes and stop tumor growth. However, the efficacy of previously identified small molecular drugs, which can induce the “anti-Warburg effect” was limited by their limited delivery to GSC, rapid systemic clearance and dose-limiting toxicities. To solve these problems, we integrated previously described “anti-Warburg drugs” with iron oxide nanocarrier platforms developed in our lab. Dichloroacetate (DCA) is a small molecule inhibitor of pyruvate dehydrogenase kinase (PDK) which can effectively inhibit the Warburg effect and induce a metabolic switch from aerobic glycolysis to the TCA cycle, thereby inducing GBM differentiation. Conjugating DCA to nanoparticles significantly limits its delivery to the normal brain, increases its blood half live and improves accumulation in tumors through the enhanced permeability and retention (EPR) effect.

To further increase tumor-specific activity, in this invention, we attached DCA to nanoparticle through a peptide linker, which can be cleaved by matrix metalloproteinase 14 (MMP-14), an enzyme highly overexpressed in most GBM, but not normal brain cells. Thus, we developed novel MMP-14 activatable anti-Warburg nanoparticles, which inhibit GBM proliferation through GSC differentiation. These nanoparticles are activated in presence of MMP-14 to release DCA, which causes a metabolic switch in GSCs, deprives them of ATP and induces differentiation to cells of neuronal lineages. In to addition, the iron oxide nanoparticle moiety of a drug according this invention allows for real-time monitoring of drug accumulation in GBM with MR imaging, which can be used to monitor and titrate tumor drug delivery. The MMP-14 activatable anti-Warburg nanoparticles of this invention hold the potential to substantially reducing the tumorigenic potential of GCS, and thereby, ultimately improve survival of GBM-bearing patients.

The anti-Warburg iron oxide nanoparticles according to this invention, stop GBM growth by differentiating glioblastoma stem cells (GSCs). These nanoparticles will selectively release the anti-Warburg drug dichloroacetate (DCA) within the tumor tissue, thereby inducing selective differentiation of GSCs and GBM tumor cells into neurons and astrocytes. DCA is a small molecular inhibitor of pyruvate dehydrogenase kinase and can switch metabolism from glycolysis to oxidative phosphorylation. The presence of DCA inhibits aerobic glycolysis or the Warburg effect in tumors. Furthermore, DCA reduces glucose uptake and causes tumors to undergo differentiation. However, a major clinical challenge with all small molecular inhibitors is the associated dose-limiting toxicities. Conjugation to a nanoparticle backbone limits DCA accumulation in the normally functioning brain while enhancing DCA payload in tumors due to the enhanced permeability and retention effect. Additionally, in the embodiments of this invention, the nanoparticles are linked to a matrix metalloproteinase-14 (MMP-14)-cleavable peptide, which activates the drug in tumor tissues, while not affecting normal organs. MMP-14 is abundant in GBMs and is vital for GBM growth, invasion, and metastases. Studies show that MMP-14 mRNA is present in 100% of glioblastomas, but only in 22% of anaplastic astrocytomas, and is not at all present in low-grade astrocytomas or the normal brain. Given that this new theranostic drug could selectively treat tumors by activating only in the presence of tumor enzymes, we scan avoid toxic side effects to normal tissues. Furthermore, the nanoparticle backbone will enable in vivo, real-time monitoring of tumor drug accumulation and distribution with magnetic resonance (MR) imaging.

In one example, embodiments of the invention could be varied by having the DCA drug be linked to other nanoparticles than ferumoxytol. Ferumoxytol nanoparticles could be linked to an alternate drug to DCA, which can also induce tumor cell differentiation.

What is claimed is:

1. A matrix metalloprotease (MMP) activatable conjugate to inhibit glioblastoma (GBM) proliferation through glioma tumor cell and glioma stem cell differentiation, comprising:
(a) a dichloroacetate (DCA) for inducing glioblastoma (GMB) differentiation;
(b) a nanoparticle comprising a magnetic resonance imaging (MRI) contrast agent, the MRI contrast agent comprising an ion; and
(c) an MMP cleavable peptide linking the DCA and the nanoparticle.

2. The matrix metalloprotease (MMP) activatable conjugate as set forth in claim 1, wherein the dichloroacetate (DCA) is a therapeutic drug.

3. The matrix metalloprotease (MMP) activatable conjugate as set forth in claim 1, wherein the ion is a gadolinium, iron, platinum, manganese, copper, gold or barium.

4. The matrix metalloprotease (MMP) activatable conjugate as set forth in claim 1, wherein the ion is an iron oxide.

5. The matrix metalloprotease (MMP) activatable conjugate as set forth in claim 1, wherein the iron oxide is a superparamagnetic iron oxide (SIPO) or an ultra superparamagnetic iron oxide (uSIPO).

6. The matrix metalloprotease (MMP) activatable conjugate as set forth in claim 1, wherein the conjugate does not comprise folate.

7. The matrix metalloprotease (MMP) activatable conjugate as set forth in claim 1, wherein the conjugate does not comprise a vascular disrupting agent (VDA).
